(19) United States
(12) Patent Application Publication
(10) Pub. No.: US 2015/0158020 A1
(43) Pub. Date: Jun. 11, 2015

(54) SYNTHESIS OF ZEOLITES USING AN ORGANOAMMONIUM COMPOUND

(71) Applicant: UOP LLC, Des Plaines, IL (US)

(72) Inventors: Christopher P. Nicholas, Evanston, IL (US); Mark A. Miller, Niles, IL (US)

(21) Appl. No.: 14/552,654

(22) Filed: Nov. 25, 2014

Related U.S. Application Data
(60) Provisional application No. 61/914,838, filed on Dec. 11, 2013.

(51) Int. Cl. B01J 29/80; C07D 211/06; C07D 295/037; C07D 471/10; B01J 29/70; C07D 207/04

(52) U.S. Cl. CPC B01J 29/80; B01J 29/70; B01J 29/7034; C07D 207/04; C07D 295/037; C07D 471/10; C07D 211/06

(57) ABSTRACT

A method for synthesizing a zeolite includes the steps of: (a) preparing an aqueous mixture comprising water, a substituted hydrocarbon and an amine; (b) reacting the aqueous mixture; (c) obtaining a solution comprising an organoammonium product; (d) forming a reaction mixture including reactive sources of M, Al, Si, optionally seeds of a layered material L, and the solution, wherein M is a metal; and (e) heating the reaction mixture to form the zeolite. The substituted hydrocarbon can be an α,ω-dihalogen substituted alkane, and the amine is preferably essentially incapable of undergoing pyramidal inversion.

### CROSS-REFERENCE TO RELATED APPLICATION

This application claims priority from Provisional Application No. 61/914,838 filed Dec. 11, 2013, the contents of which are hereby incorporated by reference in its entirety.

### BACKGROUND OF THE INVENTION

This invention relates to a process for preparing quaternary ammonium salts and a process for preparing crystalline aluminosilicate or silicate compositions including the quaternary ammonium salts. The process involves first forming an aqueous phase solution of a quaternary ammonium salt from suitable reagents such as a di-substituted alkane and an amine. The pre-reacted quaternary ammonium salt solution may then be incorporated into a zeolite reaction mixture containing sources of aluminum, silicon, and optionally other reagents, and the resultant mixture reacted at a temperature and for a time to crystallize the aluminosilicate or silicate composition.

Zeolites are crystalline aluminosilicate or silicate compositions which are microporous and which are formed from corner sharing AlO₂ and SiO₂ tetrahedra. Numerous zeolites, both naturally occurring and synthetically prepared, are used in various industrial processes. Synthetic zeolites are prepared via hydrothermal synthesis employing suitable sources of Si, Al and structure directing agents such as alkali metals, alkaline earth metals, amines, or organoammonium cations. The structure directing agents reside in the pores of the zeolite and are largely responsible for the particular structure that is ultimately formed. These species balance the framework charge associated with aluminum and can also serve as space fillers. Zeolites are characterized by having pore openings of uniform dimensions, having a significant ion exchange capacity, and being capable of reversibly desorbing an adsorbed phase which is dispersed throughout the internal voids of the crystal without significantly displacing any atoms which make up the permanent zeolite crystal structure. Zeolites can be used as catalysts for hydrocarbon conversion reactions, which can take place on outside surfaces as well as on internal surfaces within the pore.

Synthesis of zeolitic materials often relies on the use of organoammonium templates known as organic structure directing agents (OSDAs). While simple OSDAs such as tetramethylammonium, tetraethylammonium and tetrapropylammonium are commercially available, often, OSDAs are complicated molecules that are difficult and expensive to synthesize; however, their importance lies in their ability to impart aspects of their structural features to the zeolite to yield a desirable pore structure. For example, the synthesis of N,N,N,-trimethylmyrtanylammonium derivatives allowed the synthesis of CIT-1, a member of the CON zeotype (Lobo and Davis J. Am. Chem. Soc. 1995, 117, 3766-79), the synthesis of a methyl substituted N,N,N′,N′-tetraethylbicyclo[2.2.2]oct-7-ene-2,3,5,6-dipyrrolidinium diiodide enabled the synthesis of ITQ-37, the member of the ITV zeotype (Sun, et. al. Nature, 2009, 458, 1154-7) and synthesis of the trans isomer of N,N-diethyl-2-methyldecahydroquinolinium iodide (Elomari, et. al. Micro. Meso. Mater. 2009, 118, 325-33) allowed synthesis of SSZ-56, the member of the SFS zeotype.

The art clearly shows that use of complex organoammonium SDAs often results in new zeolitic materials. However, the synthesis of these complicated organoammonium compounds is quite lengthy and requires many steps, often in an organic solvent, thereby hindering development of the new zeolitic material. Frequently, even for simple, commercially available OSDAs, the OSDA is the most costly ingredient used in synthesizing zeolitic materials. Consequently, it would be economically advantageous to synthesize new zeolites from either commercially available organoammonium SDAs or SDAs which may be readily synthesized from commercially available starting materials.

The complicated OSDA(s) discussed previously were synthesized ex-situ and added to the reaction mixture at several points. However, one drawback of ex-situ synthesis is the process is typically carried out in the presence of an organic solvent, which necessitates at least one undesirable purification step to recover the SDA from the unwanted organic material. Alternatively, the OSDA(s) may be prepared in-situ, wherein the precursor materials may be added to hydrothermal synthesis reaction mixture either separately or together. For example, the inventors have discovered that the SDA precursor combination of an amine and an alkylatable organic, such as a dibromoalkane, may be added directly to the cooled reaction mixture during the synthesis of UZM-39 and UZM-44. However, this approach presents two difficulties for scale-up. First, the use of cooling prior to the addition of the OSDA or SDA precursors is both energy intensive (and therefore costly) and difficult to implement on a large scale. Second, without cooling, it is more difficult to work with SDA precursor materials with odor and flashpoint concerns, (e.g., the amine, N-methylpyrrolidine) than with an organoammonium SDA prepared in an aqueous ex-situ solution offering low odor and flashpoint as described herein.

Therefore, what is needed in the art is a method of producing a variety of organoammonium compounds for use as SDAs in zeolytic materials synthesis which overcomes the problems of purification for ex-situ synthesis and cost/safety concerns associated with in-situ synthesis.

### SUMMARY OF THE INVENTION

The present invention discloses a process for preparing a pre-reacted aqueous solution of substituted hydrocarbons and amines incapable of undergoing pyramidal inversion, which overcomes the aforementioned difficulties. The inventors have made the surprising discovery that a substituted hydrocarbon and amine may be reacted in an aqueous solution at (or slightly above) room temperature (20° C.-80° C.) to yield an aqueous solution comprising the OSDA. This solution may then be used without purification in the synthesis of zeolites. This procedure thereby allows the preparation of SDAs, such as unusual quaternary ammonium salts, from readily available starting reagents in a facile and practical manner.

OSDAs prepared by the methods of the present invention are in aqueous solution and do not pose odor and flashpoint concerns. The result is the unprecedented ability to remove the cooling step typically required in the preparation of in situ zeolite reaction mixtures and to avoid purification steps such as evaporation of organic solvent typically required in ex-situ preparation methods.

In one aspect, the invention provides a method for synthesizing a zeolite. The method includes the steps of: (a) preparing an aqueous mixture comprising water, a substituted hydrocarbon and an amine other than trimethylamine wherein the amine is a tertiary amine or secondary amine having 9 or less carbon atoms and being essentially incapable of undergoing pyramidal inversion, or combinations thereof; (b) reacting the aqueous mixture; (c) obtaining a solution comprising an organoammonium product; (d) forming a reaction mixture including reactive sources of M, Al, Si, optionally seeds of a layered material L, and the solution, wherein M is a metal; and (e) heating the reaction mixture to form the zeolite. In one version of the method, the step of reacting the aqueous mixture occurs at a temperature between 20° C. and 100° C. In another version of the method, the organoammonium product is a structure directing agent.

In another version of the method, the substituted hydrocarbon is selected from the group consisting of halogen substituted alkanes having from 2 to 8 carbon atoms, α,ω-dihalogen substituted alkanes having from 3 to 6 carbon atoms, di-halogen substituted alkanes having from 3 to 8 carbon atoms, tri-halogen substituted alkanes having from 3 to 8 carbons and combinations thereof.

In another version of the method, the substituted hydrocarbon is a halogen substituted alkane selected from the group consisting of bromoethane, iodoethane, chloropropane, bromopropane, iodopropane, chlorobutane, bromobutane, iodobutane, chloropentane, bromopentane, iodopentane, chlorohexane, bromohexane, iodohexane, 1-chloro-2-phenylethane, 1-bromo-2-phenylethane, and 1-iodo-2-phenylethane.

In another version of the method, the a α,ω-dihalogen substituted alkanes having from 3 to 6 carbon atoms selected from the group consisting of 1,3-di-halo-propane, 1,4-di-halo-butane, 1,5-di-halo-pentane, 1,6-di-halo-hexane, 1,3-di-halo-propane, 1,4-di-halo-butane, 1,5-di-halo-pentane, 1,6-di-halo-hexane; a dihalogen substituted alkane having from 3 to 6 carbon atoms selected from the group consisting of 1,2-di-halo-propane, 1,3-di-halo-butane, 1,3-di-halo-pentane, 1,4-di-halo-pentane, 2,4-di-halo-pentane, 1,5-di-halo-hexane, 1,4-di-halo-hexane, 1,3-di-halo-hexane, 2,4-di-halo-hexane, and 2,5-di-halo-hexane; a tri-halogen substituted alkane having from 3 to 8 carbon atoms selected from the group consisting of 1,2,3-tri-halo-propane, 1,2,4-tri-halo-butane, 1,2,3-tri-halo-butane, 1,3,5-tri-halo-pentane, 1,2,4-tri-halo-pentane, 1,2,3-tri-halo-pentane, 1,3,6-tri-halo-hexane, 1,2,4-tri-halo-hexane, 1,2,5-tri-halo-hexane, 1,2,6 tri-halo-hexane, 1,3,4-tri-halo-hexane, and 1,3,5-tri-halo-hexane; and any combination thereof; wherein the halogen substitution may be chloro, bromo or iodo.

In another version of the method, the amine is a tertiary amine having 9 or fewer carbon atoms. In another version of the method, the amine is selected from the group consisting of 1-methylaziridine, 1-ethylpyrrolidine, 1-methylpyrrolidine, 1-ethylazetidine, 1-methylazetidine, 1-methylhomopiperidine, 1-(2-hydroxyethyl)pyrrolidine, 1-methyl-4-piperidone, 1,3,3-trimethylpyrrolidine, 3-methyl-1-thia-3-azacyclopentane, 1-methylpiperidine, 1,2,2,6-tetramethylpiperidine, 9-methyl-9-azabicyclo[3.3.1]nonane, 1-methyloctahydro-1H-cyclopenta[B]pyridine, 4-methyl-1-oxa-4-azacyclohexane, 4-ethyl-1-oxa-4-azacyclohexane, 1-alkylpyrrolidines, 1-alkylpiperidines, and 4-alkylmorpholines and combinations thereof, and the secondary amine having 9 or fewer carbons are selected from the group comprising cyclopentylamine, methylcyclopentylamine, hexamethyleneimine (homopiperidine), 1-oxa-4-azacyclohexane, decahydroquinoline, 2-methylazetidine, 2-methylhomopiperidine, 4-piperidone, 2-piperidone, pyrrolidine, 3,3-dimethylpyrrolidine, 2-methylpyrrolidine, 3-methylpyrrolidine, 2-hydroxymethylpyrrolidine, 3-hydroxymethylpyrrolidine, piperidine, 2-methylpiperidine, 3-methylpiperidine, 4-methylpiperidine, 2,6-dimethylpiperidine, 3,5-dimethylpiperidine, octahydroindolizine, 2-methyloctahydroindolizine, pyrrolidines, piperidines, morpholines and combinations thereof.

In another version of the method, step (d) comprises forming a first mixture of the reactive sources of M, Al, Si, and the optional seeds of a layered material L, and adding the solution to the first mixture without cooling the first mixture.

In another aspect, the invention provides a method for synthesizing an organoammonium compound. The method includes the steps of: preparing an aqueous mixture comprising water, a substituted hydrocarbon and an amine other than trimethylamine wherein the amine is a tertiary or secondary amine having 9 or less carbon atoms and being essentially incapable of undergoing pyramidal inversion, or combinations thereof; reacting the aqueous mixture; obtaining a solution comprising the organoammonium compound; and wherein the mixture and the solution are essentially free of aluminum and silicon. In one version of the method, the step of reacting the aqueous mixture occurs at a temperature from about 20° C. to about 100° C., and for a time from about 0.5 hours to about 48 hours. In another version of the method, the organoammonium product is used as a structure directing agent in the synthesis of a zeolite. In another version of the method, the substituted hydrocarbon is selected from the group consisting of halogen substituted alkanes having from 2 to 8 carbon atoms, α,ω-dihalogen substituted alkanes having from 3 to 6 carbon atoms, di-halogen substituted alkanes having from 3 to 8 carbon atoms, tri-halogen substituted alkanes having from 3 to 8 carbons and combinations thereof.

In another version of the method, the substituted hydrocarbon is α,ω-dihalogen substituted alkane. In another version of the method, the α,ω-dihalogen substituted alkane is selected from the group consisting of selected from the group consisting of 1,3-dichloropropane, 1,4-dichlorobutane, 1,5-dichloropentane, 1,6-dichlorohexane, 1,3-dibromopropane, 1,4-dibromobutane, 1,5-dibromopentane, 1,6-dibromohexane, 1,3-diiodopropane, 1,4-diiodobutane, 1,5-diiodopentane, 1,6-diiodohexane and combinations thereof. In another version of the method, the tertiary amine having 9 or fewer carbon atoms and being essentially incapable of undergoing pyramidal inversion is selected from the group consisting of 1-alkylpyrrolidines, 1-alkylpiperidines, 4-alkylmorpholines, and combinations thereof and the secondary amine having 9 or fewer carbon atoms and being essentially incapable of undergoing pyramidal inversion are selected from the group consisting of pyrrolidines, piperidines, morpholines, and combinations thereof. In another version of the method, the tertiary amine having 9 or fewer carbon atoms is selected from the group comprising 1-methylaziridine, 1-ethylpyrrolidine, 1-methylpyrrolidine, 1-ethylazetidine, 1-methylazetidine, 1-methylhomopiperidine, 1-(2-hydroxyethyl)pyrrolidine, 1-methyl-4-piperidone, 1,3,3-trimethylpyrrolidine, 3-methyl-1-thia-3-azacyclopentane, 1-methylpiperidine, 1,2,2,6-tetramethylpiperidine, 9-methyl-9-azabicyclo[3.3.1]nonane, 1-methyloctahydro-1H-cyclopenta[B]pyridine, 4-methyl-1-oxa-4-azacyclohexane, 4-ethyl-1-oxa-4-azacyclohexane, 1-alkylpyrrolidines, 1-alkylpiperidines, and 4-alkylmorpholines and combinations thereof. In another version of the method, the secondary amine having 9 or fewer carbons is selected from the group comprising cyclopentylamine, methylcyclopentylamine, hexamethyleneimine (homopiperidine), 1-oxa-4-azacyclohexane, decahydroquinoline, 2-methylazetidine, 2-methylhomopiperidine, 4-piperidone, 2-piperidone, pyrrolidine, 3,3-dimethylpyrrolidine, 2-methylpyrrolidine, 3-methylpyrrolidine, 2-hydroxymethylpyrrolidine, 3-hydroxymethylpyrrolidine, piperidine, 2-methylpiperidine, 3-methylpiperidine, 4-methylpiperidine, 2,6-dimethylpiperidine, 3,5-dimethylpiperidine, octahydroindolizine, 2-methyloctahydroindolizine, pyrrolidines, piperidines, morpholines and combinations thereof. In yet another aspect, the substituted hydrocarbon is a α,ω-dihalogen substituted alkane having from 3 to 6 carbon atoms selected from the group consisting of 1,3-di-halo-propane, 1,4-di-halo-butane, 1,5-di-halo-pentane, 1,6-di-halo-hexane; a dihalogen substituted alkane having from 3 to 6 carbon atoms selected from the group consisting of 1,2-di-halo-propane, 1,3-di-halo-butane, 1,3-di-halo-pentane, 1,4-di-halo-pentane, 2,4-di-halo-pentane, 1,5-di-halo-hexane, 1,4-di-halo-hexane, 1,3-di-halo-hexane, 2,4-di-halo-hexane, and 2,5-di-halo-hexane; a tri-halogen substituted alkane having from 3 to 8 carbon atoms selected from the group consisting of 1,2,3-tri-halo-propane, 1,2,4-tri-halo-butane, 1,2,3-tri-halo-butane, 1,3,5-tri-halo-pentane, 1,2,4-tri-halo-pentane, 1,2,3-tri-halo-pentane, 1,3,6-tri-halo-hexane, 1,2,4-tri-halo-hexane, 1,2,5-tri-halo-hexane, 1,2,6 tri-halo-hexane, 1,3,4-tri-halo-hexane, and 1,3,5-tri-halo-hexane; and any combination thereof; wherein the halogen substitution may be chloro, bromo or iodo.

In another aspect, the invention provides a zeolite prepared by a process comprising the steps of: (a) preparing an aqueous mixture comprising water, a di-substituted hydrocarbons and an amine other than trimethylamine wherein the amine is a tertiary or secondary amine having 9 or less carbon atoms and being essentially incapable of undergoing pyramidal inversion, or combinations thereof; (b) reacting the aqueous mixture; (c) obtaining a solution comprising a structure directing agent; (d) forming a reaction mixture including reactive sources of M, Al, Si, seeds of a layered material L, and the solution, wherein is a metal; and (e) heating the reaction mixture to form the zeolite. In one version of the process, an organic solvent is not used in obtaining the structure directing agent.

It is therefore an advantage of the present invention to provide a system and method for preparing structure directing agents in an aqueous reaction mixture wherein the structure directing agents are prepared in the absence of Si and Al reactive sources. Furthermore, the aqueous mixture is capable of forming an organoammonium halogen salt such as a bromide salt, in order to ultimately provide a solution including a quaternary organoammonium compound. The organoammonium bromide salt can be ion-exchanged, either by reaction with Ag₂O or by anion exchange resins to yield the hydroxide form of the organoammonium or used as the halogen salt directly. Finally, the resultant organoammonium compound can be used for the synthesis of a zeolite.

These and other features, aspects, and advantages of the present invention will become better understood upon consideration of the following detailed description, drawings and appended claims.

### BRIEF DESCRIPTION OF THE DRAWINGS

FIG. 1A illustrates several examples of substituted amine compounds undergoing pyramidal inversion.

FIG. 1B illustrates several examples of quaternary ammonium compounds produced by the methods herein.

FIG. 2 shows an x-ray diffraction (XRD) spectrum for a high TUN content UZM-39 prepared according to the methods of the present invention.

### DETAILED DESCRIPTION OF THE INVENTION

The present invention deals with an aqueous process for preparing an organoammonium structure directing agent (OSDA) that overcomes many of the typical problems associated with OSDA synthesis and subsequent zeolite synthesis. Embodiments of the present invention cover methods for synthesis of OSDAs from a variety of starting materials.

In one aspect of the present invention, the OSDAs are prepared from a substituted hydrocarbon and an amine. Suitable substituted hydrocarbons include halogen substituted alkanes having between 2 and 8 carbon atoms, α,ω-dihalogen substituted alkanes having between 3 and 6 carbon atoms, di-halogen substituted alkanes having between 3 and 8 carbon atoms, tri-halogen substituted alkanes having between 3 and 8 carbons and combinations thereof. Halogens include chlorine, bromine and iodine. In an aspect, the halogen is chlorine or iodine. In another aspect, the halogen is bromine. In an aspect, the identity of the halogen substitutions on a substituted hydrocarbon may be all different, all the same, or any combination thereof. Suitable halogen substituted alkanes having from 2 to 8 carbon atoms include, but are not limited to, bromoethane, iodoethane, chloropropane, bromopropane, iodopropane, chlorobutane, 1-bromobutane, 2-bromobutane, iodobutane, 1-bromo-2-methylpropane, 2-bromo-2-methylpropane, chloropentane, bromopentane, iodopentane, 2-bromopentane, chlorohexane, bromohexane, iodohexane, benzyl bromide, 1-chloro-2-phenylethane, 1-bromo-2-phenylethane, and 1-iodo-2-phenylethane. α,ω-dihalogen substituted alkanes having between 3 and 6 carbon atoms may be selected from the group consisting of 1,3-dichloropropane, 1,4-dichlorobutane, 1,5-dichloropentane, 1,6-dichlorohexane, 1,3-dibromopropane, 1,4-dibromobutane, 1,5-dibromopentane, 1,6-dibromohexane, 1,3-diiodopropane, 1,4-diiodobutane, 1,5-diiodopentane, 1,6-diiodohexane and combinations thereof. Di-halogen substituted alkanes having between 3 and 8 carbon atoms suitably include, but are not limited to, 1,2-dibromopropane, 1,3-dibromobutane, 1,3-dibromopentane, 1,4-dibromopentane, 2,4-dibromopentane, 1,5-dibromohexane, 1,4-dibromohexane, 1,3-dibromohexane, 2,4-dibromohexane, 2,5-dibromohexane, 2,5-dibromo-3-methylhexane, 2,5-dibromo-3,3-dimethylhexane, 1,4-dibromo-2-ethylbutane, and 1,2-dibromo-2-phenylethane. Halogen substitutions may be chlorine, bromine or iodine, but are illustrated for bromine. In an aspect, the two halogen substitutions may be the same or different. Tri-halogen substituted alkanes having between 3 and 8 carbons suitably include, but are not limited to, 1,2,3-tribromopropane, 1,2,4-tribromobutane, 1,2,3-tribromobutane, 1,3,5-tribromopentane, 1,2,4-tribromopentane, 1,2,3-tribromopentane, 1,3,6-tribromohexane, 1,2,4-tribromohexane, 1,2,5-tribromohexane, 1,2,6-tribromohexane, 1,3,4-tribromohexane, and 1,3,5-tribromohexane. Halogen substitutions may be chlorine, bromine or iodine, but are illustrated for bromine. In an aspect, the identity of the three halogen substitutions on the substituted hydrocarbon may be all different, all the same, or any combination thereof. In an aspect, the mole ratio of the amine to the substitution is from about 1:1 to about 2:1 and is preferably from about 1:1 to about 1.5:1. Typically, the mole ratio of amine to substitution is approximately 1. Thus, when butylbromide is used as the substituted hydrocarbon, approximately 1 equivalent of amine is typically used, whereas when 1,4-dibromobutane is used as the substituted hydrocarbon, approximately 2 equivalents of amine are typically used.

In one aspect of the present invention, the OSDAs are prepared from a di-substituted hydrocarbons and an amine. Examples of suitable di-substituted hydrocarbons include α,ω-dihalogen substituted alkanes having between 3 and 6 carbon atoms selected from the group consisting of 1,3-dichloropropane, 1,4-dichlorobutane, 1,5-dichloropentane, 1,6-dichlorohexane, 1,3-dibromopropane, 1,4-dibromobutane, 1,5-dibromopentane, 1,6-dibromohexane, 1,3-diiodopropane, 1,4-diiodobutane, 1,5-diiodopentane, 1,6-diiodohexane and combinations thereof.

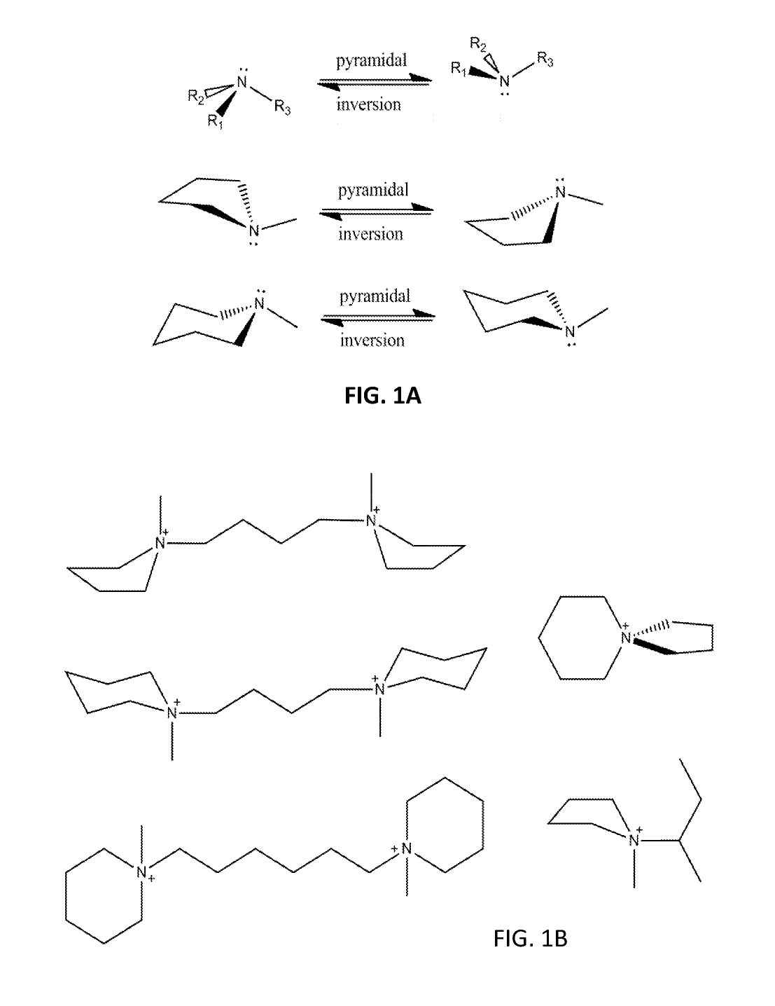

Suitable amines include those for which at least one conformer is essentially incapable of undergoing pyramidal inversion. The IUPAC definition of pyramidal inversion is given as, “a polytopal rearrangement in which the change in bond directions to a three-coordinate central atom having a pyramidal arrangement of bonds (tripodal arrangement) causes the central atom (apex of the pyramid) to appear to move to an equivalent position on the other side of the base of the pyramid. If the three ligands to the central atom are different pyramidal inversion interconverts enantiomers.” The tripodal nature of many nitrogen compounds result in the ability of these compounds to undergo pyramidal inversion. Typically, the energy barrier to inversion is low for unconstrained molecules. For example, ammonia (NH₃) has an inversion barrier of 24.5 kJ mol⁻¹, with an observed inversion frequency of about 2.4*10¹⁰ s⁻¹, dimethylamine has an inversion barrier of 18 kJ mol⁻¹, triisopropylamine has an inversion barrier of 6-8 kJ mol⁻¹ and dimethylethylamine has an inversion barrier of 22 kJ mol⁻¹. However, inversion barrier energy can become very high when the nitrogen substituents are part of a small ring or other rigid molecule as in the case of 1-methylpyrrolidine. Molecules defined as essentially incapable of undergoing pyramidal inversion have an inversion barrier energy of at least about 28 kJ mol⁻¹ and more preferably of at least about 30 kJ mol⁻¹. A discussion of pyramidal inversion may be found in Rauk, A., et al., (1970), Pyramidal Inversion. Angew. Chem. Int. Ed. Engl., 9: 400-414, with further discussion specifically for amines found in “Inorganic Chemistry” edited by Arnold F. Holleman, et al., Academic Press, 2001. Furthermore, FIGS. 1A-B illustrate several examples of substituted amine compounds undergoing pyramidal inversion and examples of quaternary ammonium compounds formed from amines which are essentially incapable of undergoing pyramidal inversion. Molecules may exist in many conformers or folding patterns. For example, it is well known that both chair and boat forms of cyclohexane exist and interconvert between the two different conformers. In an aspect of the invention, at least one conformer of the amine is essentially incapable of undergoing pyramidal inversion.

Suitable amines include tertiary amines other than trimethylamine having 9 or fewer carbon atoms and being essentially incapable of undergoing pyramidal inversion, and secondary amines having 9 or fewer carbon atoms and being essentially incapable of undergoing pyramidal inversion. Tertiary amines having 9 or fewer carbon atoms include 1-methylaziridine, 1-ethylpyrrolidine, 1-methylpyrrolidine, 1-ethylazetidine, 1-methylazetidine, 1-methylhomopiperidine, 1-(2-hydroxyethyl)pyrrolidine, 1-methyl-4-piperidone, 1,3,3-trimethylpyrrolidine, 3-methyl-1-thia-3-azacyclopentane, 1-methylpiperidine, 1,2,2,6-tetramethylpiperidine, 9-methyl-9-azabicyclo[3.3.1]nonane, 1-methyloctahydro-1H-cyclopenta[B]pyridine, 4-methyl-1-oxa-4-azacyclohexane, 4-ethyl-1-oxa-4-azacyclohexane, 1-alkylpyrrolidines, 1-alkylpiperidines, and 4-alkylmorpholines. Tertiary amines having 9 or fewer carbon atoms and being essentially incapable of undergoing pyramidal inversion may be selected from the group consisting of 1-alkylpyrrolidines, 1-alkylpiperidines, 4-alkylmorpholines, and combinations thereof.

Pyrrolidine is a 5-membered heterocycle with an N atom; 1-alkylpyrrolidines include 1-alkylpyrrolidine, 1-alkyl-2-alkylpyrrolidine, 1-alkyl-3-alkyl-pyrrolidine, 1-alkyl-2-alkyl-2-alkylpyrrolidine, 1-alkyl-2-alkyl-3-alkylpyrrolidine, 1-alkyl-2-alkyl-4-alkylpyrrolidine, 1-alkyl-2-alkyl-5-alkylpyrrolidine, 1-alkyl-3-alkyl-3-alkylpyrrolidine, 1-alkyl-3-alkyl-4-alkylpyrrolidine, 1-alkyl-3-alkyl-5-alkylpyrrolidine, and 1-alkyl-4-alkyl-4-alkylpyrrolidine where alkyl has the formula CₘH₂ₘ₊₁ and m is in the range from 1 to 4.

Piperidine is a 6-membered heterocycle with an N atom; 1-alkylpiperidines include 1-alkyl piperidine, 1-alkyl-2-alkyl piperidine, 1-alkyl-3-alkyl-piperidine, 1-alkyl-4-alkyl-piperidine, 1-alkyl-2-alkyl-2-alkyl piperidine, 1-alkyl-2-alkyl-3-alkyl piperidine, 1-alkyl-2-alkyl-4-alkyl piperidine, 1-alkyl-2-alkyl-5-alkyl piperidine, 1-alkyl-2-alkyl-6-alkyl piperidine, 1-alkyl-3-alkyl-3-alkyl piperidine, 1-alkyl-3-alkyl-4-alkyl piperidine, 1-alkyl-3-alkyl-5-alkyl piperidine, 1-alkyl-3-alkyl-6-alkyl piperidine, and 1-alkyl-4-alkyl-4-alkyl piperidine where alkyl has the formula CₘH₂ₘ₊₁ and m is in the range from 1 to 4.

Morpholine is a 6-membered heterocycle with an N atom and an O atom; 4-alkylmorpholines include 4-alkyl morpholine, 4-alkyl-2-alkyl morpholine, 4-alkyl-3-alkyl-morpholine, 4-alkyl-2-alkyl-2-alkyl morpholine, 4-alkyl-2-alkyl-3-alkyl morpholine, 4-alkyl-2-alkyl-5-alkyl morpholine, 4-alkyl-2-alkyl-6-alkyl morpholine, 4-alkyl-3-alkyl-3-alkyl morpholine, 4-alkyl-3-alkyl-5-alkyl morpholine, and 4-alkyl-3-alkyl-6-alkyl morpholine, where alkyl has the formula CₘH₂ₘ₊₁ and m is in the range from 1 to 4. Alkyl groups in the previous classes can be the same, different or any combination thereof at the different carbon atoms at which they are substituted.

Secondary amines having 9 or fewer carbons include cyclopentylamine, methylcyclopentylamine, hexamethyleneimine (homopiperidine), 1-oxa-4-azacyclohexane, decahydroquinoline, 2-methylazetidine, 2-methylhomopiperidine, 4-piperidone, 2-piperidone, pyrrolidine, 3,3-dimethylpyrrolidine, 2-methylpyrrolidine, 3-methylpyrrolidine, 2-hydroxymethylpyrrolidine, 3-hydroxymethylpyrrolidine, piperidine, 2-methylpiperidine, 3-methylpiperidine, 4-methylpiperidine, 2,6-dimethylpiperidine, 3,5-dimethylpiperidine, octahydroindolizine, 2-methyloctahydroindolizine, pyrrolidines, piperidines, and morpholines. Suitable amines may include a single amine or a combination of one or more. Secondary amines having 9 or fewer carbon atoms and being essentially incapable of undergoing pyramidal inversion may be selected from the group consisting of pyrrolidines, piperidines, morpholines, and combinations thereof.

Pyrrolidine is a 5-membered heterocycle with an N atom; pyrrolidines include pyrrolidine, 2-alkylpyrrolidine, 3-alkyl-pyrrolidine, 2-alkyl-2-alkylpyrrolidine, 2-alkyl-3-alkylpyrrolidine, 2-alkyl-4-alkylpyrrolidine, 2-alkyl-5-alkylpyrrolidine, 3-alkyl-3-alkylpyrrolidine, 3-alkyl-4-alkylpyrrolidine, 3-alkyl-5-alkylpyrrolidine, and 4-alkyl-4-alkylpyrrolidine where alkyl has the formula CₘH₂ₘ₊₁ and m is in the range from 1 to 4.

Piperidine is a 6-membered heterocycle with an N atom; piperidines include piperidine, 2-alkyl piperidine, 3-alkyl-piperidine, 4-alkyl-piperidine, 2-alkyl-2-alkyl piperidine, 2-alkyl-3-alkyl piperidine, 2-alkyl-4-alkyl piperidine, 2-alkyl-5-alkyl piperidine, 2-alkyl-6-alkyl piperidine, 3-alkyl-3-alkyl piperidine, 3-alkyl-4-alkyl piperidine, 3-alkyl-5-alkyl piperidine, 3-alkyl-6-alkyl piperidine, and 4-alkyl-4-alkyl piperidine where alkyl has the formula CₘH₂ₘ₊₁ and m is in the range from 1 to 4. Morpholine is a 6-membered heterocycle with an N atom and an O atom; morpholines include morpholine, 2-alkyl morpholine, 3-alkyl-morpholine, 2-alkyl-2-alkyl morpholine, 2-alkyl-3-alkyl morpholine, 2-alkyl-5-alkyl morpholine, 2-alkyl-6-alkyl morpholine, 3-alkyl-3-alkyl morpholine, 3-alkyl-5-alkyl morpholine, and 3-alkyl-6-alkyl morpholine, where alkyl has the formula CₘH₂ₘ₊₁ and m is in the range from 1 to 4. Alkyl groups in the previous classes can be the same, different or any combination thereof at the different carbon atoms at which they are substituted.

Table 1 provides examples of Molecules generally incapable of undergoing pyramidal inversion.

TABLE 1

| | Inversion Barrier |
|---|---|
| Molecule Name | (kJ mol⁻¹) |
| N-methylhomopiperidine | 28-29 |
| 1-methyl-4-piperidone | 30.7 |
| trimethylamine | 31-35 |
| 1,3,3-trimethylpyrrolidine | 31 |
| N-methylpyrrolidine | 31-35 |
| 3-methyl-1-thia-3-azacyclopentane | 33 |
| 9-methyl-9-azabicyclo[3.3.1]nonane | 34 |
| N-methyl piperidine (equatorial) | 36.4 |
| 1,2,2,6-tetramethylpiperidine (axial) | 38 |
| 2-methyl-dihydro-2-azaphenalene | 40.5 |
| methylazetidine | 42 |
| 1,2,2,6-tetramethylpiperidine (equitorial) | 46 |
| 4-methyl-1-oxa-4-azacyclohexane | 48 |
| 2-methyl-1-oxa-2-azacyclohexane (equitorial) | 57 |
| 2-methyl-1-oxa-2-azacyclopentane | 65 |
| methylaziridine | 80-90 |

In an aspect, the substituted hydrocarbon is a α,ω-dihalogen substituted alkanes having from 3 to 6 carbon atoms selected from the group consisting of 1,3-di-halo-propane, 1,4-di-halo-butane, 1,5-di-halo-pentane, 1,6-di-halo-hexane; a dihalogen substituted alkane selected from the group consisting of 1,2-di-halo-propane, 1,3-di-halo-butane, 1,3-di-halo-pentane, 1,4-di-halo-pentane, 2,4-di-halo-pentane, 1,5-di-halo-hexane, 1,4-di-halo-hexane, 1,3-di-halo-hexane, 2,4-di-halo-hexane, and 2,5-di-halo-hexane; a tri-halogen substituted alkane having from 3 to 8 carbon atoms selected from the group consisting of 1,2,3-tri-halo-propane, 1,2,4-tri-halo-butane, 1,2,3-tri-halo-butane, 1,3,5-tri-halo-pentane, 1,2,4-tri-halo-pentane, 1,2,3-tri-halo-pentane, 1,3,6-tri-halo-hexane, 1,2,4-tri-halo-hexane, 1,2,5-tri-halo-hexane, 1,2,6 tri-halo-hexane, 1,3,4-tri-halo-hexane, and 1,3,5-tri-halo-hexane; and any combination thereof; wherein the halogen substitution may be chloro, bromo or iodo.

In a typical method for preparing an OSDA of the present invention, a substituted hydrocarbon is added to water to form a mixture. The amine may then be added and the reaction mixture stirred until a solution containing the SDA is observed. If the solution is cooled to room temperature, the SDA product is stably maintained as an aqueous solution for later use.

In certain embodiments, the SDA precursor reagents (e.g., the substituted alkane and amine) may be added separately or together to form the SDA reaction mixture at a number of points in the process. The precursors may be reacted together at temperatures ranging from about 0° C. to about 125° C. Preferably the precursors are reacted at about room temperature or at a slightly elevated temperature such as temperatures ranging from about 5° C. to about 100° C. More preferably, the precursors are reacted at temperatures from about 20° C. to about 80° C. Other known techniques require the use of purification steps such as distillation, crystallization, chromatography and removal of a component via vacuum. A benefit of the instant method is that the solution of the organoammonium compound is prepared without additional purification steps occurring prior to use of the SDA solution. Some small laboratory scale procedures may involve removal of unreacted reactants, see Example 8 or 9, i.e., however, in commercial embodiments the reaction is most likely to react to completion, see Example 6, i.e. Ion-exchange as described below does not purify the solution, but simply converts halide anions for hydroxide ions and thus is not a purification step. The resulting SDA solution may be cooled to room temperature or used as is. However, no purification steps occur prior to use of the solution.

The methods of the present invention may be carried out in preparation of microporous crystalline zeolites such as UZM-39 and UZM-44, described in US 2013/0164213 and U.S. Pat. No. 8,623,32, respectively. UZM-39 and -44 have an empirical composition (in the as synthesized and anhydrous basis) expressed by:

NaₙMₘ^{k+}TₜAl₁₋ₓEₓSi_{y}O_{z}, where:
“n” is the mole ratio of Na to (Al+E) and has a value from approximately 0.05 to 0.5;
M represents a metal or metals selected from the group consisting of zinc, Group 1 (IUPAC 1), Group 2 (IUPAC 2), Group 3 (IUPAC 3), the lanthanide series of the periodic table, and any combination thereof;
“m” is the mole ratio of M to (Al+E) and has a value from 0 to 0.5;
“k” is the average charge of the metal or metals M;
T is the organic SDA or SDAs derived from reactants R, and Q;
R is an α,ω-dihalogen substituted alkane having between 3 and 6 carbon atoms;
Q is at least one neutral monoamine having 6 or fewer carbon atoms;
“t” is the mole ratio of N from the organic SDA or SDAs to (Al+E) and has a value of from 0.5 to 1.5, E is an element selected from the group consisting of gallium, iron, boron and combinations thereof;
“x” is the mole fraction of E and has a value from 0 to about 1.0;
“y” is the mole ratio of Si to (Al+E) and varies from greater than 9 to about 25; and
“z” is the mole ratio of O to (Al+E) and has a value determined by the equation:

z=(n+k·m+3+4·y)/2

Sources of aluminum include but are not limited to aluminum alkoxides, precipitated aluminas, aluminum metal, aluminum hydroxide, sodium aluminate, aluminum salts and alumina sols. Specific examples of aluminum alkoxides include, but are not limited to aluminum sec-butoxide and aluminum ortho isopropoxide. Sources of silica include but are not limited to tetraethylorthosilicate, colloidal silica, precipitated silica and alkali silicates. Sources of sodium include but are not limited to sodium hydroxide, sodium bromide, sodium aluminate, and sodium silicate.

As described in US 2013/0164213 and U.S. Pat. No. 8,623,321, T is the organic SDA or SDAs derived from reactants R and Q where R is an α,ω-dihalogen substituted alkane having between 3 and 6 carbon atoms and Q comprises at least one neutral monoamine having 6 or fewer carbon atoms. R may be an α,ω-dihalogen substituted alkane having between 3 and 6 carbon atoms selected from the group consisting of 1,3-dichloropropane, 1,4-dichlorobutane, 1,5-dichloropentane, 1,6-dichlorohexane, 1,3-dibromopropane, 1,4-dibromobutane, 1,5-dibromopentane, 1,6-dibromohexane, 1,3-diiodopropane, 1,4-diiodobutane, 1,5-diiodopentane, 1,6-diiodohexane and combinations thereof. As described in US 2013/0164213 and U.S. Pat. No. 8,623,321, Q comprises at least one neutral monoamine having 6 or fewer carbon atoms such as 1-ethylpyrrolidine, 1-methylpyrrolidine, 1-ethylazetidine, 1-methylazetidine, triethylamine, diethylmethylamine, dimethylethylamine, trimethylamine, dimethylbutylamine, dimethylpropylamine, dimethylisopropylamine, methylethylpropylamine, methylethylisopropylamine, dipropylamine, diisopropylamine, cyclopentylamine, methylcyclopentylamine, hexamethyleneimine. Q may comprise combinations of multiple neutral monoamines having 6 or fewer carbon atoms.

L comprises at least one seed of a layered zeolite. Suitable seed zeolites are layered materials that are microporous zeolites with crystal thickness in at least one dimension of less than about 30 to about 50 nm. The microporous materials have pore diameters of less than about 2 nm. The seed of a layered zeolite is of a different zeotype than the UZM-39 coherently grown composite being synthesized. Examples of suitable layered materials include but are not limited to UZM-4M (see U.S. Pat. No. 6,776,975), UZM-5 (see U.S. Pat. No. 6,613,302), UZM-8 (see U.S. Pat. No. 6,756,030), UZM-8HS (see U.S. Pat. No. 7,713,513), UZM-26 (see U.S. Patent Application Publication No. 2010/0152023), UZM-27 (see U.S. Pat. No. 7,575,737), BPH, FAU/EMT materials, *BEA or zeolite Beta, members of the MWW family such as MCM-22P and MCM-22, MCM-36, MCM-49, MCM-56, ITQ-1, ITQ-2, ITQ-30, ERB-1, EMM-10P and EMM-10, SSZ-25, and SSZ-70 as well as smaller microporous materials such as PREFER (pre ferrierite), NU-6 and the like.

M represents at least one exchangeable cation of a metal or metals from Group 1 (IUPAC 1), Group 2 (IUPAC 2), Group 3 (IUPAC 3) or the lanthanide series of the periodic table and or zinc. Specific examples of M include but are not limited to lithium, potassium, rubidium, cesium, magnesium, calcium, strontium, barium, zinc, yttrium, lanthanum, gadolinium, and mixtures thereof. Reactive sources of M include, but are not limited to, the group consisting of halide, nitrate, sulfate, hydroxide, or acetate salts. E is an element selected from the group consisting of gallium, iron, boron and combinations thereof, and suitable reactive sources include, but are not limited to, boric acid, gallium oxyhydroxide, gallium nitrate, gallium sulfate, ferric nitrate, ferric sulfate, ferric chloride and mixtures thereof.

For UZM-39 and UZM-44, the reaction mixture containing reactive sources of the desired components can be described in terms of molar ratios of the oxides by the formula:

a−bNa₂O:bM_{n/2}O:cRO:dQ:1−eAl₂O₃:eE₂O₃:fSiO₂:gH₂O, where:
“a” has a value of about 10 to about 30;
“b” has a value of 0 to about 30;
“c” has a value of about 1 to about 10;
“d” has a value of about 2 to about 30;
“e” has a value of 0 to about 1.0;
“f” has a value of about 30 to about 100; and
“g” has a value of about 100 to about 4000.

Additionally, when synthesizing UZM-39, in the reaction mixture is from about 1 to about 10 wt.-% of seed zeolite L based on the amount of SiO₂ in the reaction, (e.g., if there is 100 g of SiO₂ in the reaction mixture, from about 1 to about 10 g of seed zeolite L would be added).

The examples demonstrate a specific order of addition leading to the reaction mixtures from which the OSDAs described herein are formed. However, as there are a number of starting materials, many orders of addition are possible.

Other zeolites may also be synthesized from the organoammonium solutions described herein. In an aspect, the invention provides a zeolite prepared by a process comprising the steps of: (a) preparing an aqueous mixture comprising water, a substituted hydrocarbon and an amine; (b) reacting the aqueous mixture; (c) obtaining a solution comprising an organoammonium compound; (d) forming a reaction mixture including reactive sources of M, Al, Si, optional seeds of a layered material L, and the solution, wherein M is a metal; and (e) heating the reaction mixture to form the zeolite. In one version of the process, an organic solvent is not used in obtaining the structure directing agent. In another version of the process, the amine is essentially incapable of undergoing pyramidal inversion.

The zeolites prepared from the OSDAs of the process of this invention can be used as a catalyst or catalyst support in various hydrocarbon conversion processes. Hydrocarbon conversion processes are well known in the art and include cracking, hydrocracking, alkylation of aromatics or isoparaffins, isomerization of paraffin, olefins, or poly-alkylbenzene such as xylene, trans-alkylation of poly-alkybenzene with benzene or mono-alkybenzene, disproportionation of mono-alkybenzene, polymerization, reforming, hydrogenation, dehydrogenation, transalkylation, dealkylation, hydration, dehydration, hydrotreating, hydrodenitrogenation, hydrodesulfurization, methanation and syngas shift process. Preferred hydrocarbon conversion processes are those in which hydrogen is a component such as hydrotreating or hydrofining, hydrogenation, hydrocracking, hydrodenitrogenation, hydrodesulfurization, etc.

### EXAMPLES

In order to more fully illustrate the invention, the following examples are set forth. It is to be understood that the examples are only by way of illustration and are not intended as a limitation on the broad scope of the invention as set forth in the appended claims.

### Example 1

17.74 g water was weighed into a Teflon bottle. Under constant stirring, 8.18 g of 1,4-dibromobutane (99% purity) was added to the water. Two phases were observed with the lower density water phase on top. Next, 9.56 g of N-methylpyrrolidine (97% purity) was added to the two-phase mixture. Upon addition of the N-methylpyrrolidine, the mixture turned cloudy. After approximately 15 minutes, there were two phases including a clear viscous lower phase. The temperature of the mixture rose. After 30 minutes, a yellow solution was observed and the temperature of the mixture further increased to between about 40° to about 50° C. After one hour, the yellow solution cooled to room temperature. The product is stable as a yellow solution. ¹³C nuclear magnetic resonance (NMR) was used to confirm that the product was a 1,4-bis(N-methylpyrrolidinium)butane solution. Peaks were observed at 20.6, 21.4, 48.5, 63.4 and 64.5 ppm with respect to tetramethylsilane with integral ratios of 2:4:2:2:4 respectively. Resonances for the starting material N-methylpyrrolidine were present at 23.6, 40.9 and 55.3 ppm with integral ratios of 2:1:2, respectively. ¹³C NMR indicates that the dibromide solution is stable for greater than 2 years without degradation of the diquaternary salt.

### Example 2

13.1 g water was weighed into a Teflon bottle. Under constant stirring, 7.26 g of 1,4-dibromobutane (99% purity) was added to the water. Two phases were observed with the lower density water phase on top. Next, 5.84 g of N-methylpyrrolidine (97% purity) was added to the two-phase mixture. Upon addition of the N-methylpyrrolidine, the mixture turned cloudy. After approximately 15 minutes, there were two phases including a clear viscous lower phase. The temperature of the mixture rose. After 30 minutes, a yellow solution was observed and the temperature of the mixture further increased to between about 40° to about 50° C. After one hour, the yellow solution cooled to room temperature. The product was stable as a yellow solution. ¹³C nuclear magnetic resonance (NMR) was used to confirm the product was a 1,4-bis(N-methylpyrrolidinium)butane solution with no excess N-methylpyrrolidine by observation of peaks at 20.6, 21.4, 48.5, 63.4 and 64.5 ppm with respect to tetramethylsilane with integral ratios of 2:4:2:2:4 respectively and a lack of peaks at 23.6, 40.9 and 55.3 ppm.

### Example 3

18.46 g water was weighed into a Teflon bottle which was placed into a glass beaker on a hot plate. Under constant stirring, 8.90 g of 1,5-dibromopentane (99% purity) was added to the water. Two phases were observed with the lower density water phase on top. Next, 9.56 g of N-methylpyrrolidine (97% purity) was added to the two-phase mixture. Upon addition of the N-methylpyrrolidine, the mixture turned cloudy and two phases are observed. The mixture was then heated to about 60° C. After about 15 minutes, a yellow solution was observed. Upon cooling to room temperature, the product was stable as a yellow solution. ¹³C nuclear magnetic resonance (NMR) was used to confirm the product was a 1,5-bis(N-methylpyrrolidinium)pentane solution which also contained N-methylpyrrolidine.

### Example 4

12.24 g water was weighed into a Teflon bottle which was placed into a glass beaker on a hot plate. Under constant stirring, 7.03 g of 1,5-dibromopentane (99% purity) was added to the water. Two phases were observed with the lower density water phase on top. Next, 5.21 g of N-methylpyrrolidine (97% purity) was added to the two-phase mixture. Upon addition of the N-methylpyrrolidine, the mixture turned cloudy and two phases were observed. The mixture was then heated to about 60° C. After about 15 minutes, a yellow solution was observed. Upon cooling to room temperature, the product was stable as a yellow solution. ¹³C nuclear magnetic resonance (NMR) was used to confirm the product was a 1,5-bis(N-methylpyrrolidinium)butane solution with no N-methylpyrrolidine.

### Example 5

970 g of water was weighed into a 2 L Teflon bottle and the bottle placed in a 4 L beaker. Under constant stirring, 396.75 g 1,4 dibromobutane, 99% was added to the water. Then, 296.36 g N-methylpyrrolidine, 97% was added. Approximately 1.5 L “cold” tap water was placed in the 4 L beaker surrounding the Teflon bottle to help disperse the exotherm of reaction. After about 15 minutes, the mixture started to turn yellow and heat up, so ice was added to the bath. The exotherm was warm enough to form condensation, but the mixture did not reach a boil. Diquat formation was complete in about an hour.

### Example 6

836.8 g water was weighed into a 2 L Teflon bottle and the bottle placed in a 4 L beaker. Under constant stirring, 431.82 g 1,4 dibromobutane, 99% was added to the water. Then 400.68 g N-methylpiperidine, 99% was added. Approximately 1.5 L “cold” tap water was placed in the 4 L beaker surrounding the Teflon bottle to help disperse the exotherm of reaction. This mixture goes to a white single phase in about 3 hours. Overnight, the preparation became a light orange, yellowish solution. Product weight was 1666 g.

### Example 7

852.4 g water was weighed into a 2 L Teflon bottle and the bottle placed in a 4 L beaker. Under constant stirring, 489.7 g 1,5 dibromopentane, 97% was added to the water. Then 362.7 g N-methylpyrrolidine, 97% was added. Approximately 1.5 L “cold” tap water was placed in the 4 L beaker surrounding the Teflon bottle to help disperse the exotherm of reaction. After about 15 minutes, the mixture started to turn yellow and heat up, so ice was added to the bath. The exotherm was relatively strong. Product weight is 1697 g.

### Example 8

874.8 g water was weighed into a 2 L Teflon bottle and the bottle placed in a 4 L beaker. Under constant stirring, 474.1 g 1,5 dibromopentane, 97% was added to the water. Then 400.7 g N-methylpiperidine, 99% was added. Approximately 1.5 L “cold” tap water was placed in the 4 L beaker surrounding the Teflon bottle to help disperse the exotherm of reaction. After stirring overnight, the template solution appeared to be done. Given time, about 40 g 1,5 dibromopentane settled out and was removed using a separatory funnel. Analysis of the isolated yellow solution shows 51.0% water.

### Example 9

908.97 g water was weighed into a 2 L Teflon bottle and the bottle placed in a 4 L beaker. Under constant stirring, 508.29 g 1,6 Dibromohexane, 96% was added to the water. Then 400.7 g N-methylpiperidine, 99% was added. Approximately 1.5 L tap water was placed in the 4 L beaker surrounding the Teflon bottle to help control the heat of reaction. The solution was allowed to mix over the weekend where the white slurry present on Friday turned into a yellow solution. Some unreacted 1,6 dibromohexane separated out on the bottom and was removed with a separatory funnel.

### Example 10

422.44 g water was weighed into a 2 L Teflon bottle and the bottle placed in a 4 L beaker. Under constant stirring, 218.1 g 1,4 dibromobutane, 99% was added to the water. Then 204.34 g 4-Methylmorpholine, 99% was added. Approximately 1.5 L tap water was placed in the 4 L beaker surrounding the Teflon bottle to help control the heat of reaction. Low heat, approximately 50° C., was used to warm up the mixture and stirring was continued until a yellow solution was formed and no clear additional phase was present. ¹³C NMR of the solution shows a ratio of 1 mole methylmorpholine to 2.83 moles 1,4-bis(4-methylmorpholinium)butane dibromide.

### Example 11

13.98 g 2-bromobutane (98% purity) was weighed into a Teflon bottle. Under constant stirring, 22.75 g of water was added. Next, 8.78 g of N-methylpyrrolidine (97% purity) was added to the mixture. Upon addition of the N-methylpyrrolidine, the mixture turned cloudy white. After approximately 15 minutes, the mixture was transferred to a 125 mL Parr autoclave and the autoclave placed in a 125° C. oven for 3 hrs. The mixture was still two phases, so the autoclave was closed and placed back in the 125° C. oven for 4 more hours. A light orange solution was yielded. ¹³C nuclear magnetic resonance (NMR) was used to confirm that the product was a N-2-butyl-N-methylpyrrolidinium bromide solution also comprising N-methylpyrrolidine and butanol in a 0.45:1:0.15 ratio. Peaks were observed at 10.74, 13.98, 21.04, 21.24, 24.13, 42.09, 63.83, 64.07 and 72.55 ppm with respect to tetramethylsilane with integral ratios of 1. Resonances for the starting material N-methylpyrrolidine were present at 23.1, 40.9 and 55.3 ppm with integral ratios of 2:1:2, respectively. Resonances for butanol were present at 9.7, 21.9, 31.0 and 69.2 ppm with integral ratios of 1.

### Example 12

21.81 g of 1,4-dibromobutane (99% purity) was weighed into a Teflon bottle. Under constant stirring, 39.0 g deuterium oxide (deuterated water) was added to the dibromobutane. Two phases were observed with the lower density water phase on top. Next, 17.2 g of piperidine (99% purity) was added to the two-phase mixture. Upon addition of the piperidine, the mixture turned a cloudy white. Shortly thereafter, the temperature of the mixture rose. After a total of 90 seconds, a clear yellow solution was observed. The yellow solution was allowed to cool to room temperature. ¹³C nuclear magnetic resonance (NMR) was used to confirm that the product was a 5-azaspiro[4.5]decane bromide solution. Peaks for the spirocyclic compound were observed at 62.8, 60.6, 21.5, 21.4 and 21.1 with respect to tetramethylsilane with integral ratios of 2:2:2:2:1 respectively. Resonances for piperidinium were present at 44.8, 22.5, and 21.8 ppm with integral ratios of 2:2:1, respectively. The ratio of spirocyclic compound to piperidinium was 1:1.

### Example 13

1200 g of the 41.7 wt % solution of 1,4-bis(N-methylpyrrolidinium)butane dibromide in water from Example 5 was weighed into a round bottom flask. Under constant stirring, 306.35 g silver(I) oxide, 99%, was added. The flask was kept in the dark and allowed to mix for 40-48 hours. The resulting 1,4-bis(N-methylpyrrolidinium) butane dihydroxide solution was isolated by removing AgBr via filtration. Analysis showed 69.0% water.

### Example 14

1000 g of the 50 wt % solution of 1,4-bis(N-methylpiperidinium)butane dibromide in water from Example 6 was weighed into a round bottom flask. Under constant stirring, 285.36 g silver(I) oxide, 99%, was added. The flask was kept in the dark and allowed to mix for 40-48 hours. The resulting 1,4-bis(N-methylpiperidinium) butane dihydroxide solution was isolated by removing AgBr via filtration. Analysis showed 67.0% water.

### Example 15

1000 g of the 50 wt % solution of 1,5-bis(N-methylpyrrolidinium)pentane dibromide in water from Example 7 was weighed into a round bottom flask. Under constant stirring, 295.37 g silver(I) oxide, 99%, was added. The flask was kept in the dark and allowed to mix for 40-48 hours. The resulting 1,5-bis(N-methylpyrrolidinium)pentane dihydroxide solution was isolated by removing AgBr via filtration. Analysis shows 65.5% water and silver was reported as <0.0002 wt %.

### Example 16

1100 g of the 50 wt % 1,5-bis(N-methylpiperidinium)pentane dibromide solution in water from Example 8 was weighed into a round bottom flask. Under constant stirring, 303.64 g silver(I) oxide, 99%, was added. The flask was kept in the dark and allowed to mix for 40-48 hours. The resulting 1,5-bis(N-methylpiperidinium)pentane dihydroxide solution was isolated by removing AgBr via filtration. Analysis shows 64.5% water.

### Example 17

6.47 g Al(OH)₃ (27.9% Al by analysis) was dissolved in 224.92 g of the 31 wt % solution of 1,4-bis(N-methylpyrrolidinium)butane dihydroxide in water from Example 13. While stirring, 200 g Ludox AS-40 (18.8% Si by analysis) and 445.12 g water were added to form a solution. To a 100 g portion of this solution, 4.28 g of a LiOH.30 H₂O solution was added dropwise and mixed thoroughly before division into 4 equal parts and placed in separate 45 cc autoclaves for digestion. The resulting product from the reaction vessel digested at 160° for 14 days was identified by XRD analysis to be predominately MTW with MOR and a minor impurity present.

### Example 18

6.47 g Al(OH)₃ (27.9% Al by analysis) was dissolved in 234.04 g of the 33 wt % solution of 1,4-bis(N-methylpiperidinium)butane dihydroxide in water from Example 14. While stirring, 200 g Ludox AS-40 (18.8% Si by analysis) and 420.0 g water were added to form a solution. To a 100 g portion of this solution, 2.33 g of a 50 wt % CsOH solution was added dropwise and mixed thoroughly before division into 4 equal parts and placed in separate 45 cc autoclaves for digestion. The resulting product from the reaction vessel digested at 160° in a rotisserie oven at 15 rpm for 13 days was identified by xrd analysis to be MTW with a small amount of amorphous material present.

### Example 19

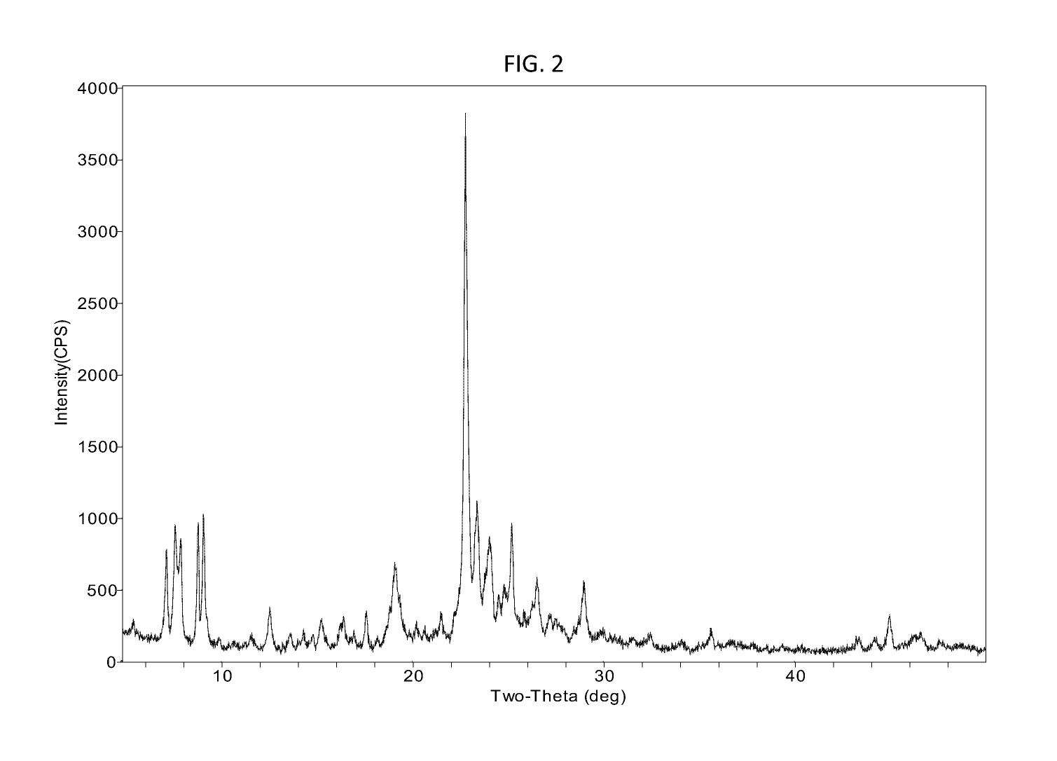

5.96 g of NaOH, (97%) was dissolved in 91.88 g water. 1.22 g Al(OH)₃, (27.9 wt.-% Al), was added to the sodium hydroxide solution. When this became a solution, 37.5 g Ludox AS-40 was added. Next, 0.30 g of the calcined, ion-exchanged layered material UZM-8 was added and the mixture was stirred vigorously for 1-2 hours before cooling. 37.3 g of the Example 1 solution was added to create the final reaction mixture. The final reaction mixture was vigorously stirred and transferred to a 300 cc stirred autoclave. The final reaction mixture was digested at 160° C. for 144 hours with stirring at 100 rpm. The product was isolated by filtration. The product was identified as a high TUN content UZM-39 by XRD; the XRD pattern is shown in FIG. 2. Analytical results showed this material to have the following molar ratios, Si/AI of 12.92, Na/AI of 0.117, N/AI of 0.915 and C/N of 7.05. Three letter codes such as TUN are assigned to specific zeolite structure types (e.g., TNU-9) by the Structure Commission of the International Zeolite Association (IZA).

Thus, the invention provides methods for synthesizing an organoammonium compound, methods for synthesizing a zeolite, and a zeolite prepared using the methods.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A method for synthesizing a zeolite, the method comprising:
(a) preparing an aqueous mixture comprising water, a substituted hydrocarbon and an amine other than trimethylamine wherein the amine is a tertiary amine or secondary amine having 9 or less carbon atoms and being essentially incapable of undergoing pyramidal inversion, or combinations thereof;
(b) reacting the aqueous mixture;
(c) obtaining a solution comprising an organoammonium product;
(d) forming a reaction mixture including reactive sources of M, Al, Si, optionally seeds of a layered material L, and the solution, wherein M is a metal; and
(e) heating the reaction mixture to form the zeolite.

2. The method of claim 1, wherein the step of reacting the aqueous mixture occurs at a temperature from about 20° C. to about 100° C.

3. The method of claim 1, wherein the organoammonium product is a structure directing agent.

4. The method of claim 1 wherein the substituted hydrocarbon is selected from the group consisting of halogen substituted alkanes having from 2 to 8 carbon atoms, α,ω-dihalogen substituted alkanes having from 3 to 6 carbon atoms, di-halogen substituted alkanes having from 3 to 8 carbon atoms, tri-halogen substituted alkanes having from 3 to 8 carbons and combinations thereof.

5. The method of claim 1 wherein the substituted hydrocarbon is a halogen substituted alkane selected from the group consisting of bromoethane, iodoethane, chloropropane, bromopropane, iodopropane, chlorobutane, bromobutane, iodobutane, chloropentane, bromopentane, iodopentane, chlorohexane, bromohexane, iodohexane, 1-chloro-2-phenylethane, 1-bromo-2-phenylethane, and 1-iodo-2-phenylethane.

6. The method of claim 1 wherein the substituted hydrocarbon is
a α,ω-dihalogen substituted alkanes having from 3 to 8 carbon atoms selected from the group consisting of 1,3-di-halo-propane, 1,4-di-halo-butane, 1,5-di-halo-pentane, 1,6-di-halo-hexane, 1,3-di-halo-propane, 1,4-di-halo-butane, 1,5-di-halo-pentane, 1,6-di-halo-hexane;
a dihalogen substituted alkane having from 3 to 8 carbon atoms selected from the group consisting of 1,2-di-halo-propane, 1,3-di-halo-butane, 1,3-di-halo-pentane, 1,4-di-halo-pentane, 2,4-di-halo-pentane, 1,5-di-halo-hexane, 1,4-di-halo-hexane, 1,3-di-halo-hexane, 2,4-di-halo-hexane, and 2,5-di-halo-hexane;
a tri-halogen substituted alkane having from 3 to 8 carbon atoms selected from the group consisting of 1,2,3-tri-halo-propane, 1,2,4-tri-halo-butane, 1,2,3-tri-halo-butane, 1,3,5-tri-halo-pentane, 1,2,4-tri-halo-pentane, 1,2,3-tri-halo-pentane, 1,3,6-tri-halo-hexane, 1,2,4-tri-halo-hexane, 1,2,5-tri-halo-hexane, 1,2,6 tri-halo-hexane, 1,3,4-tri-halo-hexane, and 1,3,5-tri-halo-hexane; and
any combination thereof;
wherein the halogen substitution may be chloro, bromo or iodo.

7. The method of claim 1, wherein the substituted hydrocarbon is α,ω-dihalogen substituted alkane.

8. The method of claim 7, wherein the α,ω-dihalogen substituted alkane is selected from the group consisting of 1,3-dichloropropane, 1,4-dichlorobutane, 1,5-dichloropentane, 1,6-dichlorohexane, 1,3-dibromopropane, 1,4-dibromobutane, 1,5-dibromopentane, 1,6-dibromohexane, 1,3-diiodopropane, 1,4-diiodobutane, 1,5-diiodopentane, 1,6-diiodohexane and combinations thereof.

9. The method of claim 1, wherein the tertiary amine having 9 or fewer carbon atoms and being essentially incapable of undergoing pyramidal inversion is selected from the group consisting of 1-alkylpyrrolidines, 1-alkylpiperidines, 4-alkylmorpholines, and combinations thereof and the secondary amine having 9 or fewer carbon atoms and being essentially incapable of undergoing pyramidal inversion is selected from the group consisting of pyrrolidines, piperidines, morpholines, and combinations thereof.

10. The method of claim 1, wherein the tertiary amine having 9 or fewer carbon atoms is selected from the group comprising 1-methylaziridine, 1-ethylpyrrolidine, 1-methylpyrrolidine, 1-ethylazetidine, 1-methylazetidine, 1-methylhomopiperidine, 1-(2-hydroxyethyl)pyrrolidine, 1-methyl-4-piperidone, 1,3,3-trimethylpyrrolidine, 3-methyl-1-thia-3-azacyclopentane, 1-methylpiperidine, 1,2,2,6-tetramethylpiperidine, 9-methyl-9-azabicyclo[3.3.1]nonane, 1-methyloctahydro-1H-cyclopenta[B]pyridine, 4-methyl-1-oxa-4-azacyclohexane, 4-ethyl-1-oxa-4-azacyclohexane, 1-alkylpyrrolidines, 1-alkylpiperidines, 4-alkylmorpholines and combinations thereof, and the secondary amine having 9 or fewer carbons is selected from the group comprising cyclopentylamine, methylcyclopentylamine, hexamethyleneimine, 1-oxa-4-azacyclohexane, decahydroquinoline, 2-methylazetidine, 2-methylhomopiperidine, 4-piperidone, 2-piperidone, pyrrolidine, 3,3-dimethylpyrrolidine, 2-methylpyrrolidine, 3-methylpyrrolidine, 2-hydroxymethylpyrrolidine, 3-hydroxymethylpyrrolidine, piperidine, 2-methylpiperidine, 3-methylpiperidine, 4-methylpiperidine, 2,6-dimethylpiperidine, 3,5-dimethylpiperidine, octahydroindolizine, 2-methyloctahydroindolizine, pyrrolidines, piperidines, morpholines and combinations thereof.

11. The method of claim 1, wherein step (d) comprises forming a first mixture of the reactive sources of M, Al, Si, and the seeds of a layered material L, and adding the solution to the first mixture without cooling the first mixture.

12. A method for synthesizing an organoammonium compound, comprising:
preparing an aqueous mixture comprising water, a substituted hydrocarbon and an amine other than trimethylamine wherein the amine is a tertiary or secondary amine having 9 or less carbon atoms and being essentially incapable of undergoing pyramidal inversion, or combinations thereof;
reacting the aqueous mixture;
obtaining a solution comprising the organoammonium compound; and
wherein the mixture and the solution are essentially free of aluminum and silicon.

13. The method of claim 12, wherein the step of reacting the aqueous mixture occurs at a temperature from about 20° C. to about 100° C., and for a time from about 0.5 hours to about 48 hours.

14. The method of claim 12, further comprising synthesizing a zeolite using the solution comprising the organoammonium compound.

15. The method of claim 12, wherein the substituted hydrocarbon is selected from the group consisting of halogen substituted alkanes having from 2 to 8 carbon atoms, α,ω-dihalogen substituted alkanes having from 3 to 6 carbon atoms, di-halogen substituted alkanes having from 3 to 8 carbon atoms, tri-halogen substituted alkanes having from 3 to 8 carbons and combinations thereof.

16. The method of claim 12, wherein the substituted hydrocarbon is an α,ω-dihalogen substituted alkane.

17. The method of claim 16, wherein the α,ω-dihalogen substituted alkane is selected from the group consisting of selected from the group consisting of 1,3-dichloropropane, 1,4-dichlorobutane, 1,5-dichloropentane, 1,6-dichlorohexane, 1,3-dibromopropane, 1,4-dibromobutane, 1,5-dibromopentane, 1,6-dibromohexane, 1,3-diiodopropane, 1,4-diiodobutane, 1,5-diiodopentane, 1,6-diiodohexane and combinations thereof.

18. The method of claim 12, wherein the tertiary amine having 9 or fewer carbon atoms and being essentially incapable of undergoing pyramidal inversion is selected from the group consisting of 1-alkylpyrrolidines, 1-alkylpiperidines, 4-alkylmorpholines, and combinations thereof and the secondary amine having 9 or fewer carbon atoms and being essentially incapable of undergoing pyramidal inversion is selected from the group consisting of pyrrolidines, piperidines, morpholines, and combinations thereof.

19. The method of claim 18, wherein the tertiary amine having 9 or fewer carbon atoms is selected from the group comprising 1-methylaziridine, 1-ethylpyrrolidine, 1-methylpyrrolidine, 1-ethylazetidine, 1-methylazetidine, 1-methylhomopiperidine, 1-(2-hydroxyethyl)pyrrolidine, 1-methyl-4-piperidone, 1,3,3-trimethylpyrrolidine, 3-methyl-1-thia-3-azacyclopentane, 1-methylpiperidine, 1,2,2,6-tetramethylpiperidine, 9-methyl-9-azabicyclo[3.3.1]nonane, 1-methyloctahydro-1H-cyclopenta[B]pyridine, 4-methyl-1-oxa-4-azacyclohexane, 4-ethyl-1-oxa-4-azacyclohexane, 1-alkylpyrrolidines, 1-alkylpiperidines, 4-alkylmorpholines and combinations thereof, and the secondary amine having 9 or fewer carbons is selected from the group comprising cyclopentylamine, methylcyclopentylamine, hexamethyleneimine, 1-oxa-4-azacyclohexane, decahydroquinoline, 2-methylazetidine, 2-methylhomopiperidine, 4-piperidone, 2-piperidone, pyrrolidine, 3,3-dimethylpyrrolidine, 2-methylpyrrolidine, 3-methylpyrrolidine, 2-hydroxymethylpyrrolidine, 3-hydroxymethylpyrrolidine, piperidine, 2-methylpiperidine, 3-methylpiperidine, 4-methylpiperidine, 2,6-dimethylpiperidine, 3,5-dimethylpiperidine, octahydroindolizine, 2-methyloctahydroindolizine, pyrrolidines, piperidines, morpholines and combinations thereof.

20. The method of claim 12 wherein the substituted hydrocarbon is a halogen substituted alkane selected from the group consisting of bromoethane, iodoethane, chloropropane, bromopropane, iodopropane, chlorobutane, bromobutane, iodobutane, chloropentane, bromopentane, iodopentane, chlorohexane, bromohexane, iodohexane, 1-chloro-2-phenylethane, 1-bromo-2-phenylethane, 1-iodo-2-phenylethane, and combinations thereof.

21. The method of claim 12 wherein the substituted hydrocarbon is
a α,ω-dihalogen substituted alkane having from 3 to 6 carbon atoms selected from the group consisting of 1,3-di-halo-propane, 1,4-di-halo-butane, 1,5-di-halo-pentane, 1,6-di-halo-hexane;
a dihalogen substituted alkane having from 3 to 8 carbon atoms selected from the group consisting of 1,2-di-halo-propane, 1,3-di-halo-butane, 1,3-di-halo-pentane, 1,4-di-halo-pentane, 2,4-di-halo-pentane, 1,5-di-halo-hexane, 1,4-di-halo-hexane, 1,3-di-halo-hexane, 2,4-di-halo-hexane, and 2,5-di-halo-hexane;
a tri-halogen substituted alkane having from 3 to 8 carbon atoms selected from the group consisting of 1,2,3-tri-halo-propane, 1,2,4-tri-halo-butane, 1,2,3-tri-halo-butane, 1,3,5-tri-halo-pentane, 1,2,4-tri-halo-pentane, 1,2,3-tri-halo-pentane, 1,3,6-tri-halo-hexane, 1,2,4-tri-halo-hexane, 1,2,5-tri-halo-hexane, 1,2,6 tri-halo-hexane, 1,3,4-tri-halo-hexane, and 1,3,5-tri-halo-hexane; and
any combination thereof;
wherein the halogen substitution may be chloro, bromo or iodo.

22. A zeolite prepared by a process comprising the steps of:
(a) preparing an aqueous mixture comprising water, a di-substituted hydrocarbon and an amine other than trimethylamine wherein the amine is a tertiary or secondary amine having 9 or less carbon atoms and being essentially incapable of undergoing pyramidal inversion, or combinations thereof;
(b) reacting the aqueous mixture;
(c) obtaining a solution comprising a structure directing agent;
(d) forming a reaction mixture including reactive sources of M, Al, Si, optionally seeds of a layered material L, and the solution, wherein M is a metal; and
(e) heating the reaction mixture to form the zeolite.

23. The zeolite of claim 22 wherein an organic solvent is not used in obtaining the structure directing agent.
